(19) United States
(12) Patent Application Publication
(10) Pub. No.: US 2012/0184006 A1
(43) Pub. Date: Jul. 19, 2012

(54) Reductase Enzymes

(75) Inventors: Robert M. Willis, Logan, UT (US); Bradley D. Wahlen, Hyrum, UT (US); Lance C. Seefeldt, Providence, UT (US); Brett M. Barney, Minneapolis, MN (US)

(73) Assignee: UTAH STATE UNIVERSITY, North Logan, UT (US)

(21) Appl. No.: 13/350,753

(22) Filed: Jan. 14, 2012

Related U.S. Application Data
(60) Provisional application No. 61/432,809, filed on Jan. 14, 2011.

(51) Int. Cl. C12P 7/02; C12N 9/02

(52) U.S. Cl. 435/155; 435/189

(57) ABSTRACT

In some embodiments, the present invention relates to isolated enzymes useful in reducing a fatty acyl-CoA to a corresponding fatty alcohol in a single biosynthetic step, polynucleotides encoding the enzymes, and methods for making and using these polynucleotides and enzymes. In some embodiments, the invention provides for isolated or recombinant enzymes capable of reducing a fatty acyl-CoA to a fatty alcohol. In still another embodiment, the invention provides for isolated or recombinant polynucleotides encoding an enzyme capable of reducing a fatty acyl-CoA to a fatty alcohol. In other embodiments, the invention provides for methods of making or using enzymes capable of reducing fatty acyl-CoA to a fatty alcohol, and methods of making using polynucleotides that encode the enzymes.

### CROSS REFERENCE TO RELATED APPLICATIONS

This application claims the benefit of U.S. provisional patent application no, 61/432,809, filed on Jan. 14, 2011, and entitled “Fatty acyl-CoA reductase enzyme from bacteria capable of reducing a fatty acyl-CoA to the corresponding alcohol,”

### STATEMENT REGARDING FEDERALLY SPONSORED RESEARCH

This invention was made with US government support under grant number 0968781 awarded by the NSF. The US government has certain rights in the invention.

### REFERENCE TO A SEQUENCE LISTING

This application includes a 7.63 KB computer readable sequence listing created on Jan. 14, 2012 using Pat-In 3.5 and entitled “P11017_{—}02_sequence_ST25,” the entire contents of which is hereby incorporated by reference herein. This application contains one or more sequence listings in paper and computer readable form; the information recorded in computer readable form is identical to the written sequence listing.

### BACKGROUND OF THE INVENTION

This invention relates to the field of molecular biology. More specifically, the invention relates to the fields of polynucleotides and enzymes.

Biological lipids serve vital functions in living systems, ranging from the separation of various cellular compartments to providing a convenient means to store reduced carbon as a long term energy reserve. One such lipid, the wax ester (R—CO—OR′), is found in cells across all three domains of life where these compounds play a variety of roles. In plants, for example, the outer surface of the epidermal cells are coated with a variety of waxes that provide a protective barrier to limit water loss, minimize damage from UV light, and limit attack from insects and pathogens. These waxes are composed of an assortment of different compounds including wax esters, very long chain fatty acids (VLCFAs), fatty aldehydes, fatty alcohols, alkanes and a range of other lipids. Each of these reduced carbon compounds are produced through a unique biological pathway, all presumed to be derived from the fatty acid pool.

The various waxes and natural hydrocarbons produced in plants and other organisms are of interest for a variety of commercial and industrial applications, from high grade lubricants, to cosmetics and soaps, as well as flavoring compounds. We propose herein the utilization of biosynthetic approaches for the production of natural biofuels to serve as substitutes for petroleum derived transportation fuels. Of these compounds, wax esters and fatty alcohols are of particular interest due to the high demand and significant markets in cosmetic, pharmaceutical, and industrial processes.

Biological production of wax esters in prokaryotes is proposed to require the activity of three enzymes: (i) reduction of a fatty acyl-CoA to the corresponding fatty aldehyde catalyzed by a fatty acyl-CoA reductase (FACoAR), (ii) reduction of the fatty aldehyde to the corresponding fatty alcohol catalyzed by a fatty aldehyde reductase (FALDR), and (iii) condensation of the fatty alcohol with a fatty acyl-CoA to yield the wax ester by a wax ester synthase/acyl-CoA: diacylglycerol acyltransferase (WS/DGAT). In contrast, in some plants, such as jojoba and Arabidopsis thaliana, it is commonly suggested that a single enzyme catalyzes both the fatty acyl-CoA reduction and fatty aldehyde reduction (FIG. 1). While a number of studies characterizing substrate ranges for the WS/DGAT have been published for both prokaryotes and eukaryotes alike, less progress has been made toward an understanding of the enzymes required to produce the requisite fatty alcohols in bacteria.

### BRIEF SUMMARY OF THE INVENTION

In one embodiment, the present invention relates to isolated enzymes capable of, and therefore useful in, reducing a fatty acyl-CoA to a corresponding fatty alcohol in a single biosynthetic step, polynucleotides encoding the enzymes, and methods for making and using these polynucleotides and enzymes. Preferably, enzymes of the present invention may be FACoAR enzymes. Optionally, enzymes of the present invention may provide for enzymes having dual reductase activity. Dual reductive activity, as used herein, means the potential to sequentially reduce at least one fatty acyl-CoA to at least one corresponding fatty aldehyde, and, to reduce the corresponding fatty aldehyde to the corresponding fatty alcohol. Optionally, enzymes of the present invention may have a higher specificity for long chain aldehydes than for shorter aldehydes.

In another embodiment, the invention provides for isolated or recombinant enzymes capable of reducing a fatty acyl-CoA to a fatty alcohol in a and having an amino acid sequence at least 50%, 60%, 70%, 80%, 90%, 95%, 98%, 99%, or 100% similar to SEQ ID NO: 1. Similarity, as used herein, refers to the chemical similarity of certain amino acids as recognized by one skilled in the art. Optionally, the enzyme may have an amino acid sequence at least 50%, 60%, 70%, 80%, 90%, 95%, 98%, 99%, or 100% identical to SEQ ID NO: 1. The enzyme of SEQ ID NO:1 was unexpectedly discovered to contain two domains. The first domain comprises amino acids 1 to 364 of SEQ ID NO:11. The second domain comprises amino acids 364 to 601. Each domain could be used to generate enzymes related to the present invention, either together or separately, using techniques standard in the art of molecular biology. For example, without limiting the invention, an enzyme comprising an amino acid sequence at least 50%, 60%, 70%, 80%, 90%, 95%, 98%, 99%, or 100% similar or identical to amino acids 364 to 601 of SEQ NO:1 may be made using techniques standard in the art, and recombined with other amino acid sequences. Alternatively, an isolated amino acid sequence at least 50%, 60%, 70%, 80%, 90%, 95%, 98%, 99%, or 100% similar or identical to amino acids 1 to 364 SEQ ID NO:1 may be made using techniques standard in the art, and recombined with other amino acid sequences. In some embodiments, any number or combination of the amino acids similar to the amino acids of SEQ ID NO:1 may be identical to the amino acids of SEQ ID NO:1. These sequences correspond to accession number YP_{—}959769 from the NCBI database.

In still another embodiment, the invention provides for isolated or recombinant polynucleotides encoding an enzyme capable of reducing a fatty acyl-CoA to a fatty alcohol, and have a nucleic acid sequence at least 50%, 60%, 70%, 80%, 90%, 95%, 98%, or 100% identical to SEQ ID NO:2.

In other embodiments, the invention provides for methods of making or using enzymes capable of reducing fatty acyl-CoA to a fatty alcohol, and methods of making using polynucleotides that encode the enzymes.

Abbreviations when used herein are as follows:

### CoA—Coenzyme A

FACoAR—Fatty acyl CoA reductase
NADPH—Reduced nicotinamide adenine dinucleotide phosphate
NADP⁺—Oxidized nicotinamide adenine dinucleotide phosphate
DTNB—5′-dithiobis(2-nitrobenzoic acid)
NTB—2-nitro-5-thiobenzoate
CMC—Critical micelle concentration
FALDR—Fatty aldehyde reductase

### BRIEF DESCRIPTION OF TEE DRAWINGS

FIG. 1 shows proposed reaction schemes for the production of alcohols by the activity of Fatty Acyl-CoA reductase enzymes. Scheme I shows a proposed two step reduction mechanism proposed for bacterial fatty acyl-CoA reductases. Scheme II shows a proposed single step reduction for the production of alcohols by, for example, some higher eukaryotes fatty acyl-CoA reductases.

FIG. 2 shows a domain arrangement of M. aquaeolei VT8 FACoAR compared to A. calcoaceticus FACoAR. Schematic domains are shown with the 661 amino acid M. aquaeolei VT8 and the 295 amino acid A. calcoaceticus sequence. Conserved regions are outlined with the C-terminal domain of the M. aquaeolei VT8 enzyme aligning with high similarity (53% identical and 74% similar) to the majority (residues 9 to 295) of the A. calcoaceticus enzyme. Denoted by * are the conserved pyridine nucleotide binding regions found in each enzyme, which have the conserved sequence GXGX(1-2X)G.

FIG. 3 shows the purification of the FACoAR. Shown is an SDS-PAGE of the purification scheme of the FACoAR from M. aquaeolei VT8 expressed from E. coli. A protein of approximately 116 kDa is obtained after each affinity purification step. Lane 1 contains the soluble cell free lysate. Lane 2 contains the elution from the amylose affinity resin. Lane 3 contains the elution from metal affinity resin charged with nickel. Lane 4 contains the elution from the G25 sephadex column. Lane 6 contains the protein standards. Protein is >95% pure following the G25 sephadex column purification.

FIG. 4 shows a TLC plate of fatty acyl-CoA reductase products of reaction. Lane 1 contains 5 μL of a palmitoleyl alcohol (10 mg/mL in hexane) standard. Lane 2 contains 5 μL of a cis-11-hexadecenal (10 mg/mL in hexane) standard. Lane 3 contains heat inactivated FACoAR from M. aquaeolei VT8 incubated with palmitoleyl-CoA and NADPH and extracted as described in the methods. Lane 4 is identical to Lane 3, except that the FACoAR was not heat inactivated. Samples were allowed to react for 1 hr with gentle shaking at room temperature before extracting with hexane, and spotting on TLC. The solvent front is the top of the image. The drawn circles indicate the point at which the samples were blotted before developing the TLC plate.

FIG. 5 shows kinetic parameters of fatty acyl-CoA reductase from M. aquaeolei VT8 indicating direct production of palmitoyl alcohol from palmitoyl CoA. The () indicate points for the DTNB assay measuring the release of free CoA. Vₘₐₓ of 115±7 nmol NTB⁻² min⁻¹ mg of protein⁻¹, apparent Kₘ of 4±0.3 μM, and a n of 2.8±0.7. The (◯) indicate points for the NADPH assay measuring the enzymatic utilization of NADPH. Vₘₐₓ of 197±8 nmol NADP⁺ min⁻¹ mg of protein⁻¹, apparent Kₘ of 4.0±0.2 μM, and n of 2.6±0.3. All kinetic parameters were calculated using a triplicate data set with Igor Pro software shown with error bars representing the standard error of the mean (SEM) fit to equation 1 in the materials and methods.

FIG. 6 shows kinetic parameters of fatty acyl-CoA reductase from M. Aquaeolei VT8 showing reactivity toward cis-11-hexadecenal. NADPH assay measuring the decrease in absorbance at 340 nm. Vₘₐₓ of 7.7±0.6 μmol min⁻¹ mg of protein⁻¹, Apparent Kₘ of 48±7 μM and a n of 2±0.8. Data shown with SEM, calculated using Visual Enzymics and Igor pro Software fit to equation 1 in the materials and methods.

FIG. 7 shows a proposed reaction mechanism for fatty acyl-CoA reductase. The top reaction shows the reduction of the fatty acyl-CoA occurring within the same active site in a two-step reduction. The bottom reaction shows the reduction of the fatty acyl-CoA at two different active sites. Active site I is shown reducing the fatty acyl-CoA to the aldehyde and releasing it. Active site 2 is shown reducing the aldehyde to the corresponding alcohol. R represents a fatty acyl Chain of varying lengths. R₂ represents the remaining portion of the NADP(H) molecule.

FIG. 8 shows an alternative proposed reaction mechanism for fatty acyl-CoA reductase. This Figure shows the reduction of the fatty acyl-CoA occurring within the same active site in a two-step reduction. The two-step chemical reduction may occur in a single biosynthetic step. The inhibition of aldehyde reduction by fatty acyl CoA is shown. The reaction could proceed through either an aldehyde intermediate or an enzyme bound intermediate for activity. Given the experimental results presented here an enzyme bound intermediate reaction is favored as no aldehyde intermediate is detectable.

FIG. 9 shows Palmitoyl CoA inhibition of fatty aldehyde reduction. The reduction of the fatty aldehyde cis-11-hexadecenal is inhibited in a competitive manner with an apparent Kᵢ of 1.9±0.26 μM Palmitoyl CoA. The (◯) denote assays performed with 0 μM palmitoyl CoA, () denote assays performed with 1 palmitoyl CoA, (□) denote assays performed with 2 μM palmitoyl CoA, and (Δ) denote assays performed with 4 μM palmitoyl CoA. Specific activities are plotted in units of nmol NADP⁺ min⁻¹ mg protein⁻¹. All assays were conducted in triplicate and are shown with error bars indicating SEM fit to equation 1 in the materials and methods section. The apparent Kᵢ was determined by fitting all four data sets to equation 2 in the materials and methods.

FIG. 10 shows a two-step dual reduction of a fatty acyl-CoA to a corresponding alcohol, carried out in a single biosynthetic step.

### DETAILED DESCRIPTION OF THE INVENTION

In one embodiment, the present invention relates to isolated enzymes useful in reducing a fatty acyl-CoA to a corresponding fatty alcohol in a single biosynthetic step, polynucleotides encoding the enzymes, and methods for making and using these polynucleotides and enzymes. Preferably, enzymes of the present invention may be FACoAR enzymes. Preferably, enzymes of the present invention may be bacterial enzymes capable of reducing a fatty acyl-CoA to a corresponding fatty alcohol in a single biosynthetic step. Optionally, enzymes of the present invention may provide for dual reductase activity. Dual reductive activity, as used herein, means the potential to sequentially reduce at least one fatty acyl-CoA to at least one corresponding fatty aldehyde, and, to reduce the corresponding fatty aldehyde to the corresponding fatty alcohol. Optionally, enzymes of the present invention may have a higher specificity for long chain aldehydes than for shorter aldehydes.

In another embodiment, the invention provides for isolated or recombinant enzymes capable of reducing a fatty acyl-CoA to a fatty alcohol in a and having an amino acid sequence at least 50%, 60%, 70%, 80%, 90%, 95%, 98%, 99%, or 100% similar to SEQ ID NO: 1.

In still another embodiment, the invention provides for isolated or recombinant polynucleotides encoding an enzyme capable of reducing a fatty acyl-CoA to a fatty alcohol, and have a nucleic acid sequence at least 50%, 60%, 70%, 80%, 90%, 95%, 98%, or 100% identical to SEQ ID NO:2.

Optionally, the discovery of enzymes capable of reducing a fatty acyl-CoA to a fatty alcohol in a single biosynthetic step eliminates the need to incorporate a second enzyme into large scale growth production of fatty alcohols. In some embodiments enzymes of the present invention may be incorporated into a model bacterial organism or host to produce large of amounts of fatty alcohols from other reduced carbon sources, such as hemi-cellulose and glucose. The enzyme may be further incorporated into an organism for the production of various wax ester compounds by a similar method, as described herein.

The FACoAR from A. aquaeolei sequence differed significantly from the FACoAR of the bacterium A. calcoaceticus that was previously known to reduce fatty acyl-CoA to the aldehyde. We describe herein a method for the purification and characterization of a unique enzyme from M. aquaeolei that has broad substrate specificity and catalyzes both reduction steps (dual reduction) from a fatty acyl-CoA substrate to the corresponding fatty alcohol.

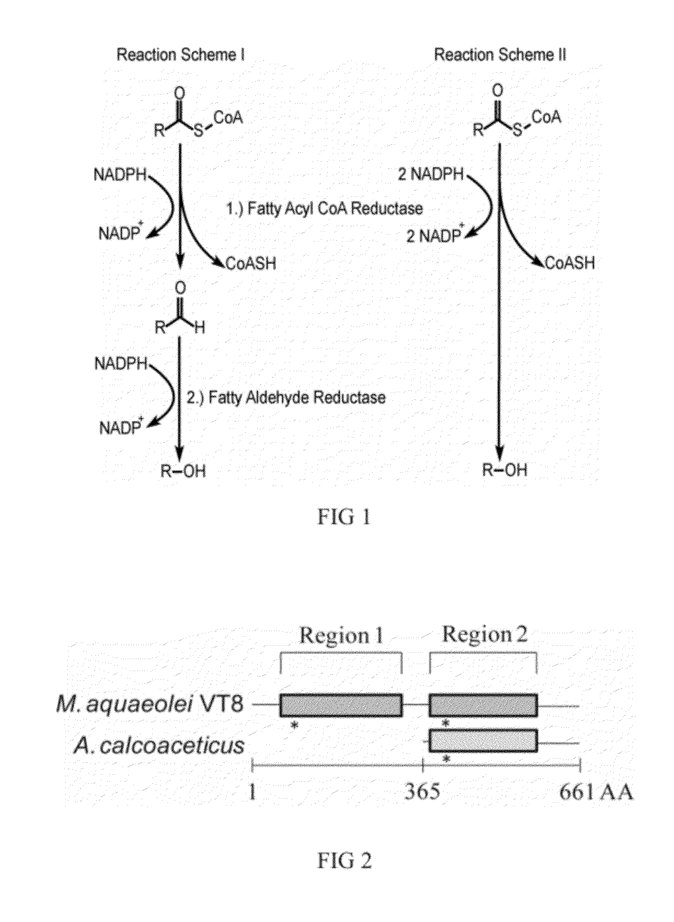

Referring now to FIG. 1, the biosynthesis of fatty alcohols occurs by one of two different mechanisms. In the plant jojoba (Simmondsia chinensis), a single FACoAR enzyme, catalyzes the four electron reduction of fatty acyl-CoA in an NADPH dependent manner to the corresponding fatty alcohol. In contrast to the jojoba FACoAR, bacterial fatty alcohol biosynthesis has been reported to occur in two steps. The first step is the two electron reduction of a fatty acyl-CoA to the corresponding fatty aldehyde and free CoA. Following this first reduction step, the fatty aldehyde must be further reduced by two electrons to the fatty alcohol either via a fitly aldehyde reductase (FALDR) or other enzymes yet to be identified.

The FACoAR from Acinetobacter calcoaceticus (ZP_{—}06058153.1) was the first FACoAR described in a bacteria, and was utilized as an initial template through which to search form similar enzyme in Marinobacter aquaeolei V7178. An enzyme from M. aquaeolei VT8 was found (YP_{—}959769) with some amino acid sequence similarity to the FACoAR from A. calcoaceticus, although the M. aquaeolei VT8 enzyme appeared to have two different domains.

Referring now to FIG. 2, the first domain on the N-terminal end of this enzyme shared a very slight sequence identity with the fatty aldehyde reductase (FALDR) previously characterized, white the second domain on the C-terminal end (beginning at approximately 375 residues and proceeding to the end of the protein) aligned with high agreement to the FACoAR from A. calcoaceticus. These similarities led to the hypothesis that the M. aquaeolei VT8 enzyme might catalyze the reduction of a fatty acyl-CoA substrate to the corresponding fatty alcohol, similar to the enzyme identified from jojoba. Although the M. aquaeolei VT8 putative FACoAR did not align well with known acyl-CoA reductases from plants, a high sequence similarity to orange of (proteins from many other bacteria was noted, including enzymes found in lipid accumulating bacteria such as Rhodococcus and several strains of Mycobacterium.

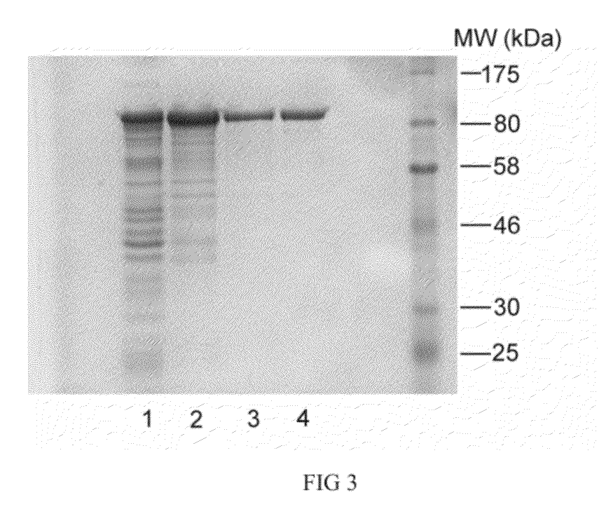

Referring now to FIG. 3, the putative FACoAR from M. aquaeolei VT8 was cloned as described in the methods section following an approach similar to that used to clone and isolate active FALDR. The only variation in the approach taken here was to transfer the final cloned fragment into an N-terminal maltose binding protein fusion vector that also contained a C-terminal 8× His-tag. This approach allowed a rapid two step purification of the enzyme using amylose affinity and metal affinity chromatography. Still referring to FIG. 3, the migration of the protein on a SDS gel agreed well with the molecular mass of 116 kDa predicted from the amino acid sequence. Earlier attempts to purify FACoAR proteins revealed solubility problems and loss of activity, which were not encountered herein, when the protein was expressed as a fusion with the maltose binding protein.

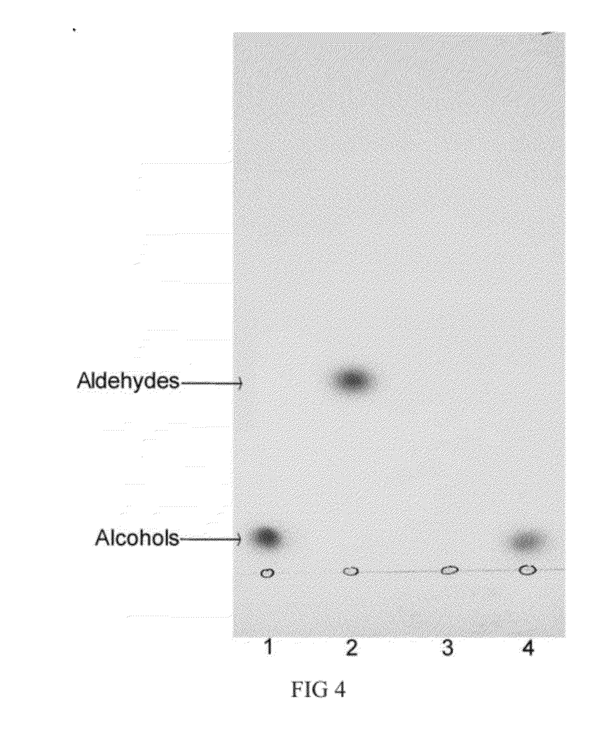

Referring now to FIG. 4, an initial assessment of protein activity was achieved by an overnight assay to determine the substrate identity and requirement for reductant, and utilized thin layer chromatography (TLC) and gas chromatography (GC) to identify the products. It was found that the only detectable product from palmitoleyl-CoA reduction was palmitoleyl alcohol, with no detection of any fatty aldehydes. Further, these initial assays revealed that the enzyme required NADPH as a reductant and only acted on the fatty acids bound to CoA. This coenzyme requirement agrees well with the other commonly known findings for the FACoAR from A. calcoaceticus, which was also found to utilize NADPH as the reductant, while eukaryotic FACoAR enzymes have been reported to use either NADPH for jojoba and honey bee (Apis mellifera) or NADH for Euglena gracilis.

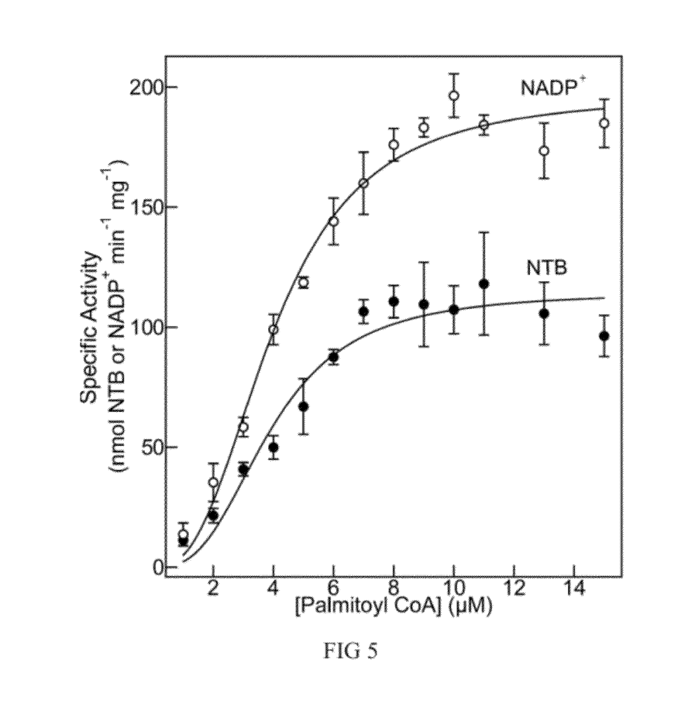

Referring now to FIG. 5, to further examine the activity of the FACoAR, a real-time assay was developed to track oxidation of NADPH. This assay complemented an assay based on the use of Ellman's reagent (DTNB) to measure the release of CoA. Using both assays, the reactions could be followed spectrophotometrically in real-time to establish the initial rates of reaction with a range of substrates. Still referring to FIG. 5, there is shown the specific activity for palmitoyl-CoA reduction versus the concentration of substrate, tracking either the formation of NTB²⁻ (the chromophore produced following reaction of DTNB with the free thiol of CoA released during the reaction) or the toss of NADPH. The data in both assays was best fit to a sigmoidal curve, indicating possible allosterism or cooperativity. Fitting the data to the Hill equation, a Kₘ of approximately 4 was μM obtained for the reduction of palmitoyl-CoA. The Vₘₐₓ was calculated to be ˜180 nmol min⁻¹ mg⁻¹ when CoA release was monitored or nearly twice that 370 nmol mg⁻¹ when NADP⁺ formation was monitored, indicating that the enzyme was catalyzing two steps in the reduction. Unexpectedly, enzymes of the present invention may have a maximal specific activity with CoA of 115 nmol of NTB/min/mg protein, which is much higher than reported specific activities of similar enzymes, which may be, for example, 0.1 mmol/min/mg protein (Reiser, S., and Somerville, C. (1997) Isolation of mutants of Acinetobacter calcoaceticus deficient in wax ester synthesis and complementation of one mutation with a gene encoding a fatty acyl coenzyme A reductase, J Bacteriol. 179, 2969-2975).

The above data indicate that the M. aquaeolei VT8 enzyme catalyzes the four electron reduction of the acyl-CoA to the alcohol, and thus the reaction is expected to pass through the corresponding 2 electron reduced aldehyde. In an attempt to trap a possible aldehyde intermediate, assays were performed in the presence of phenyl hydrazine or hydrazine using an approach similar to that taken to isolate aldehyde intermediates from crude preparations of E. gracillis (13). Assays were conducted in the same manner as described in the materials and methods section for both NADPH and DTNB spectrophotometric assays, except that 5 mM of phenyl hydrazine or hydrazine was included in the buffer to potentially trap any free aldehyde. The results showed little difference between either the initial rate of NADPH oxidation or the total quantity of NADPH consumed between the sample with hydrazine and the control without (data not shown). This indicates that either the reaction does not proceed through a free fatty aldehyde intermediate or if an intermediate is formed, it is not reactive with hydrazine in the time frame of the reaction assay.

The fatty acyl-CoA substrates used in these experiments have relatively low critical micelle concentration (CMC) values. The CMC values depend on the buffer ionic strength and protein concentration present. For fatty acyl-CoA substrates, the common generally accepted ranges for CMC values are between 10 to 40 μM. To avoid the loss of substrate availability in micelles, all specific activities were determined using fatty acyl-CoA concentrations below the published CMC values. Experiments were conducted with higher concentrations of several fatty acyl-CoA substrates, resulting in the expected lower activity with higher substrate concentration (data not shown).

The carbon length preference of potential fatty acyl-CoA substrates for the FACoAR from M. aquaeolei VT8 was also determined by examining the rates of substrate reduction for a series of acyl-CoA molecules (Table 1).

TABLE 1
Specific Activity Towards Acyl-CoA Substrates.

| | | Rate of Reaction | % Specific |
|---|---|---|---|
| | Carbon | (nmol NTB²⁻ | Activity of |
| | Chain | min⁻¹ mg | Palmitoyl- |
| Substrate | Length | protein⁻¹)^{a} | CoA^{b} |
| Octanoyl-CoA | C8 | 18.8 ± 0.83 | 34 |
| Lauroyl-CoA | C12 | 44.0 ± 6.3 | 80 |
| Myristoyl-CoA | C14 | 34.1 ± 2.1 | 58 |
| Palmitoyl-CoA | C16 | 54.7 ± 4.7 | 100 |
| Palmitoleyl-CoA | C16:1 | 53.7 ± 1.1 | 93 |
| Stearoyl-CoA | C18 | 37.6 ± 3.5 | 69 |
| Oleyl-CoA | C18:1 | 41.5 ± 3.1 | 76 |
| Arachidonoyl-CoA | C20:4 | 50.7 ± 3.66 | 93 |

^{a}Reactions were performed as described in the materials and methods section using 6 μM of the respective CoA.
^{b}All values are reported as a percent of the specific activity palmitoyl-CoA

These results show a higher rate of reduction with larger (C20:4) and smaller (C8) fatty acyl-CoA groups than was reported for the FACoAR from A. calcoaceticus, which showed low activity with substrates greater than C18 and smaller than C14.

In addition to determining whether this FACoAR from M. aquaeolei VT8 could reduce fatty acyl-CoA substrates, the ability of the enzyme to reduce fatty aldehydes to the alcohol was determined, similar to FALDR characterized from this same species. This activity was measured using the same spectrophotometric assay to track the disappearance of NADPH, but utilized a range of fatty aldehydes as substrate (Table 2).

TABLE 2
Specific Activity Towards Aldehyde Substrates.

| | Carbon | Rate of Reaction | % Specific |
|---|---|---|---|
| | Chain | (nmol NADP⁺ min⁻¹ | Activity |
| Substrate | Length | mg proten⁻¹)^{a} | of Decanal^{b} |
| Acetaldehyde | C2 | 54 ± 6.0 | <1 |
| Propanal | C3 | 43 ± 6.0 | <1 |
| Hexanal | C6 | 1540 ± 30.0 | 10 |
| Octanal | C8 | 5200 ± 1500 | 33 |
| Decanal | C10 | 15800 ± 860 | 100 |
| Dodecanal | C12 | 11700 ± 250 | 74 |
| Cis-11-hexadecenal | C16:1 | 9910 ± 600 | 63 |
| 2-Naphthaldehyde | | 5280 ± 10 | 3 |

^{a}Reactions were performed as described in the materials and methods section using 60 μm of the respective aldehyde.
^{b}All values are reported as a percent of the specific activity of decanal.

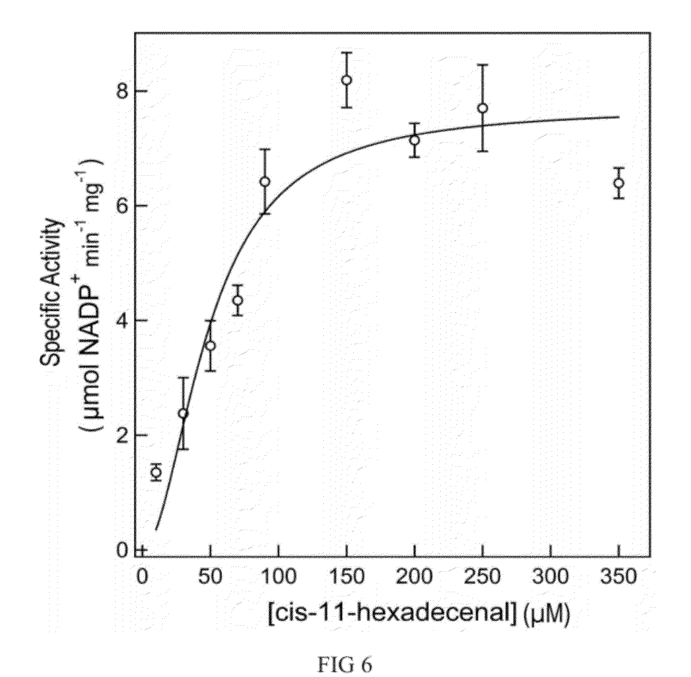

The results for the fatty aldehyde cis-11-hexadecenal are shown in FIG. 6. Again, the product was analyzed by GC and TLC analysis (not shown) to confirm that the product is the alcohol. A fit of these data to the Hill equation reveals a Kₘ˜25 μM, Vₘₐₓ of ˜10 μmol min⁻¹ mg⁻¹, with strong cooperativity n˜2. The specific activity is significantly higher than the activity found for the recently characterized FALDR enzyme from M. aquaeolei VT8. Thus, the FACoAR described here has dual reduction activity, with the potential to reduce either fatty acyl-CoA or fatty aldehyde substrates.

Referring now to FIG. 6, the FALDR shares a minimal sequence similarity with the CER4 protein from Arabidopsis, and also shares a very minimal similarity with the N-terminal domain for the FACoAR described here. The FACoAR enzyme was previously tested against a range of fatty aldehydes, and for the same substrate as shown in FIG. 6 (cis-11-hexadecenal), was found to have a Kₘ of 177 μM and a Vₘₐₓ of approximately 60 nmols min⁻¹ mg⁻¹. From these results, the FACoAR appears to have a lower Kₘ and a much higher Vₘₐₓ overall than was reported for the FALDR using the same assay. To verify that the FALDR previously isolated from M. aquaeolei VT8 did not have activity towards fatty acyl-CoA substrates, that enzyme was subjected to the same DTNB and NADPH assays described here for the FACoAR. Activity was confirmed for fatty aldehyde reduction, but no activity was detected for fatty acyl-CoA reduction, indicating a clear difference for the substrate profiles of these two enzymes.

The Vₘₐₓ for fatty aldehyde reduction for the FACoAR is also significantly higher than the Vₘₐₓ obtained for fatty acyl-CoA substrate reduction in this FACoAR. (FIG. 5), although the Kₘ for fatty acyl-CoA was significantly lower (4 μM for palmitoyl-CoA versus 25 μM for cis-11-hexadecenal). This higher rate of reduction could be a key factor in minimizing the loss of a potential intermediate aldehyde that could be toxic to the cell.

For each of the fatty acyl-CoA substrates analyzed as part of this work (see Table 1), the quantity of NADPH oxidized during the reaction was found to be approximately twice the amount of NTB²⁻ (product of the reaction with DTNB and free CoA) produced. This result indicates that any fatty aldehyde formed from the reduction of fatty acyl-CoA is immediately reduced to the aldehyde. These results can be interpreted to indicate several possible conclusions; either the enzyme contains two active sites that are close together, it contains a channel between the two active sites that retain the aldehyde during the reduction steps, or there is one active site that functions to reduce both the fatty aldehyde and the fatty acyl-CoA. This, along with the lack of an affect when hydrazine is included in the assay, indicates that the aldehyde does not diffuse away from the enzyme before being converted to the alcohol. Future work may seek to produce fragmented versions of the enzyme to test the nature of this dual step reduction further.

As shown in FIGS. 5 and 6, the activities of the FACoAR from M. aquaeolei VT8 do not follow standard Michaelis Menten kinetics. Instead, sigmoidal kinetics are observed that is best fit to the Hill equation. Cooperativity may occur with enzymes that utilize acyl-CoA substrates and are active in the synthesis of lipid compounds. 3-hydroxy-3-methyl-glutaryl-CoA(HMG-CoA) reductases from human, mammalian, and bacterial sources alike have been shown to demonstrate similar kinetic behavior.

As part of our characterization, we attempted to alleviate the sigmoidal response of activity to substrate concentration by the addition of reducing agents and detergents. In the case of another type of acyl-CoA reductase, HMG-CoA reductase, the addition of reducing agents and/or detergents could limit, but not eliminate the sigmoidal kinetics related to cooperative binding. The use of detergents and reducing agents including 5% isopropanol, Triton X-100, and β-mercaptoethanol at varying concentrations failed to alleviate the sigmoidal character of the kinetics of the M. aquaeolei VT8 FACoAR and in each case inhibited activity. This seems to indicate the potential of some inter- or intra-protein interaction that is necessary for the activity to occur. To further investigate this possibility, the protein was analyzed by size exclusion chromatography to determine the native weight of the protein.

The native weight of the protein was determined as described in the methods section using a size exclusion column. The protein flowed through the column as two separate peaks running close together. When compared to the standards the first peak ran with the void indicating aggregation of protein, however, the second peak gave an apparent size approximately four times that of the predicted monomeric state, indicating a possible tetrameric form of the enzyme. When collected fractions were run on an SDS all fractions were shown to contain the FACoAR protein, yet only the fraction corresponding to the possible tetrameric state showed activity. This indicates that the enzyme may require a higher oligomeric state for activity such as the tetrameric form. The enzyme may also take the form of a monomeric, dimeric, trimeric, or tetrameric protein form. Other oligomeric protein forms may also occur.

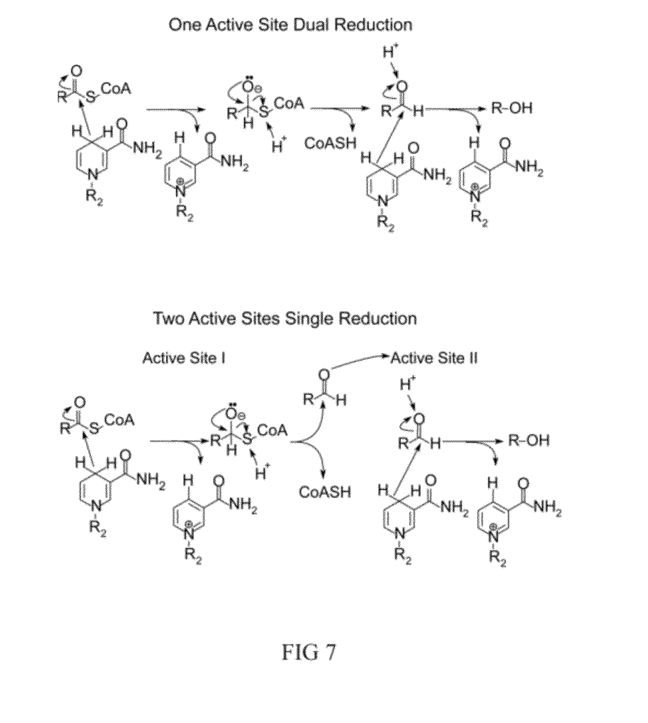

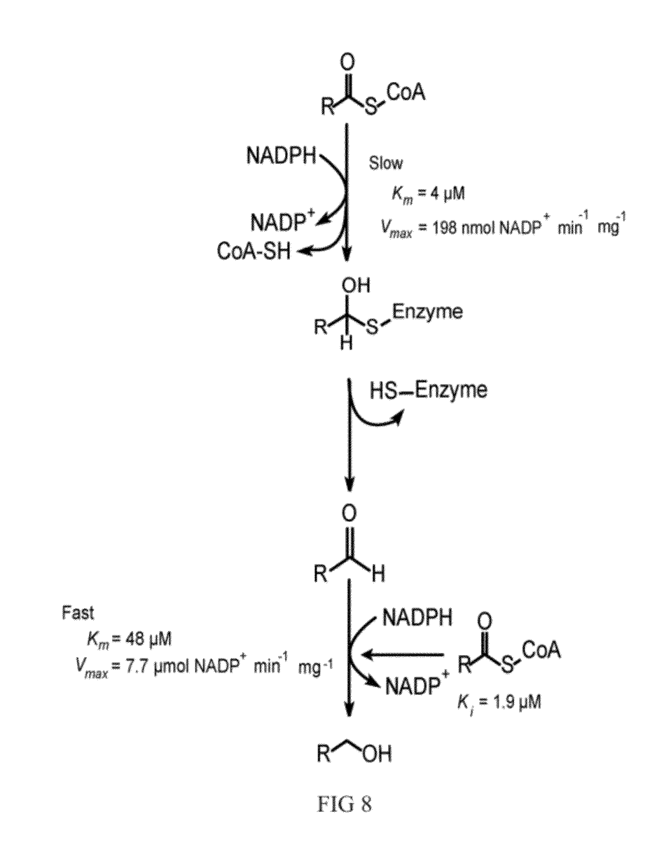

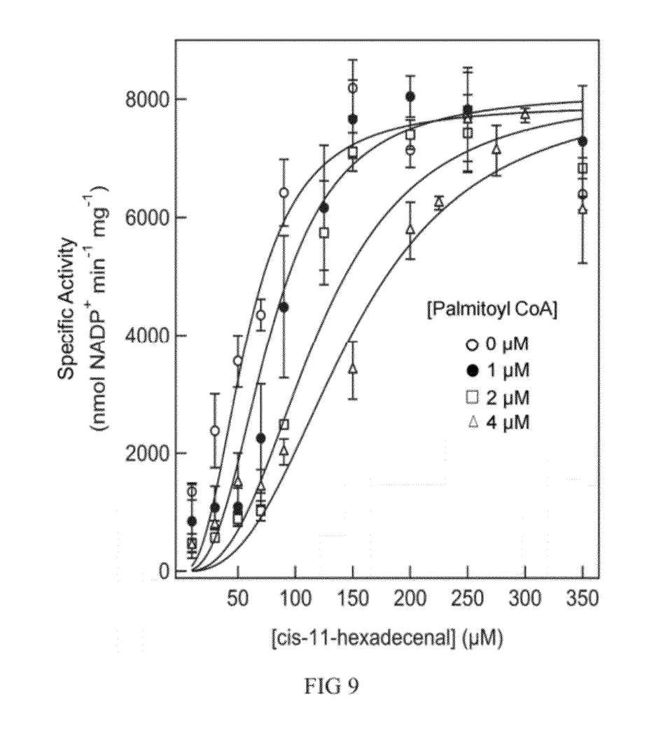

Referring now to FIG. 7, given the data that have been collected, a possible mechanism of action for this protein can be devised. Although there are no reports of enzymes fully characterized kinetically with high similarity to this enzyme, the HMG-CoA reductase discussed previously may function in a similar manner. HMG-CoA reductase is an important enzyme in the cholesterol synthase pathway. In HMG-CoA reductase the activity occurs in one active site where the CoA substrate is reduced to the alcohol completely with an aldehyde intermediate which does not leave the active site. Still referring to FIG. 7, there are shown two mechanisms of action that are possibilities given our current data. The top reaction shows a complete reduction of the fatty acyl-CoA to the fatty alcohol in a single active site, while the bottom reaction shows the aldehyde leaving one active site and going to another with little to no exposure to the solvent. Referring now to FIG. 8, there is shown an alternative proposed reaction mechanism for fatty acyl-CoA reductase. FIG. 8 shows the reduction of the fatty acyl-CoA occurring within the same active site in a two-step reduction. The two-step chemical reduction may occur in a single biosynthetic step. The inhibition of aldehyde reduction by fatty acyl CoA is shown

The sigmoidal character of the rate versus substrate concentration profile could be explained by protein-protein interactions that are likely to occur in a tetrameric form of the protein. The interacting proteins could be cooperating to allow enzymatic function much like what is seen with HMG-CoA reductase. For the latter enzyme, cleavage of sections of protein with freeze/sheer solubilization of the protein led to elimination of the sigmoidal kinetics, indicating that protein function was not affected by the loss of portions of the enzyme that could act as regulatory domains. To test whether portions of the N or C terminus of the protein are responsible for the exhibited cooperativity and necessary for catalysis, truncated versions will be constructed as part of future work. Kinetic experiments performed with the resultant enzymes could help identify the region of the protein responsible for the cooperativity in the M. aquaeolei VT8 FACoAR.

The present invention describes a bacterial enzyme from M. aquaeolei VT8 that catalyzes the reduction of fatty acyl-CoA substrates to the corresponding fatty alcohol, in contrast to other reports for bacterial enzymes that only reduce fatty acyl-CoA as far as the fatty aldehyde, in terms of function, the FACoAR from M. aquaeolei VT8 shares properties more similar to FACoAR enzymes obtained from higher eukaryotes, despite sharing little sequence similarity. The C-terminal domain of the FACoAR from M. aquaeolei VT8 shares sequence similarity with the FACoAR from A. calcoaceticus, and the N-terminus appears to have little homology to other known FACoAR enzymes. Further, the substrate specificity for the enzyme we describe is broader than the relatively narrow specificity reported for the vast majority of other FACoAR enzymes previously characterized. Homologs to this FACoAR are found in a variety of other bacteria, including other species known to accumulate wax esters, indicating that this enzyme may constitute an additional class of bacterial FACoAR enzymes in contrast to those sharing similarity with the FACoAR from A. calcoaceticus.

The above description discloses the invention including preferred embodiments thereof. The examples and embodiments disclosed herein are to be construed as merely illustrative and not a limitation of the scope of the present invention in any way. It will be obvious to those having skill in the art that many changes may be made to the details of the above-described embodiments without departing from the underlying principles of the invention.

The following examples include materials and methods used to practice some disclosed embodiments. The examples and may be useful in practicing or giving guidance in the practice of all of the various embodiments and examples disclosed herein and related to the present invention. Furthermore, aspects of each example may be, and have been, used together to practice certain embodiments of the invention described herein.

### Example 1

### Reagents

Coenzymes (NADPH, NADH, NADP⁺ and NAD⁺), various fatty acyl-CoAs, fatty aldehydes and fatty alcohols, 5,5′-dithiobis(2-nitrobenzoic acid), also referred to as Ellman's reagent or DTNB and all other reagents were purchased from Sigma-Aidrich, unless otherwise stated.

### Example 2

### Cloning and Gene Expression

The protein sequence of the fatty acyl-CoA reductase (FACoAR) from Acinetobacter calcoaceticus (ZP_{—}06058153.1) was used to perform a BLAST search of the NCBI database for a corresponding gene in Marinobacter aquaeolei VT8. The search identified a gene (YP_{—}959769.1) of 661 amino acids with approximately 50% identity (73% similarity) over a region of about 280 residues of the C-terminus of the protein. The gene was cloned by PCR from purified genomic DNA isolated from M. aquaeolei VT8 using primers (GACGAGAATT CAATTATTTC CTGACAGGCG GCACCGG) and (TCGACTCTAG ACTCCAGTAT ATCCCCCGCA TAATC) and the failsafe PCR kit (Epicenter, Madison, Wis.) and was ligated into the EcoRI and XbaI sites of a pUC derivative plasmid. Other plasmids common in the art may also be used. The entire cloned insert was sequenced to confirm no mistakes, and was moved to a pMAL-c4x plasmid derivative (New England Biolabs, Ipswich, Mass.) containing an insert for incorporation of an 8× His-tag following the in-frame insertion after the XboI site. This resulted in the final plasmid pPCRMALD8 that contains the FACoAR from M. aquaeolei VT8 with an N-terminal Maltose Binding Protein (MBP) fusion and a C-terminal His-tag. This construct contains a Factor Xa cleavage site immediately following the MBP protein to facilitate removal. The plasmid was transformed into E. coli TB1 strain for protein expression.

Protein was expressed by growing 1 L cultures in Luria Bertani broth (LB) supplemented with 100 mg/L ampicillin from an 8 mL starter culture. The culture was grown with shaking at 37° C. until the culture reached an optical density of approximately 0.6 at 600 nm. Protein expression was induced by the addition of 50 mg/L of isopropyl-β-thiogalactopyranoside (IPTG) and the culture was grown for 3-4 hours before harvesting by centrifugation. Collected cell pellets were frozen and stored at −80° C.

### Example 3

### Protein Purification

Cell pellets of approximately 4 g were resuspended in 30 mL of lysis buffer composed of 20 mM Tris-HCl pH 7.0, 50 mM NaCl, and 1 mM EDTA. The resuspended cells were placed in a 50 mL conical tube and placed in a water ice mixture to keep the cells cold during lysis. The cells were passed through a French press three times at 1000 lb/in². Whole cell lysate was centrifuged at 10,000 g for 20 min to separate the cell debris from the soluble extract.

Cell lysate was passed over an amylose column (P/N E8201L, New England Biolabs, Ipswich, Mass.) to bind the fusion protein, and washed with 3 column volumes of lysis buffer, followed by a 2 column volume wash of lysis buffer with 1M NaCl to interrupt non-specific binding. The column was then washed with 3 column volumes of equilibration buffer containing 20 mM Tris-HCl pH 7.0 and 50 mM NaCl, and the bound protein was eluted with 2 column volumes of 10 mM maltose in equilibration buffer containing 20 mM Tris-HCl pH 7.0, 50 mM NaCl. Fractions were tested for relative protein concentration by nanodrop (Thermo Scientific, Wilmington, Del.). High protein fractions were then pooled and added to a metal affinity column (P/N 17-0575-01, GE Healthcare, and Upsalla, Sweden) charged with nickel. This was followed by a wash with 3 column volumes of the same equilibration buffer. The column was then washed with 2 column volumes of 10 mM imidazole in equilibration buffer to disrupt non-specific binding. A final 3 column volume wash with equilibration buffer was performed before eluting with 2 column volumes of a 500 mM imidazole solution in equilibration buffer. Resulting fractions were analyzed on a 12% SDS-PAGE gel. Fractions with the purified protein running at about 116 kDa according the protein marker were pooled and exchanged into equilibration buffer using a G25 Sephedex column (Pharmacia Fine Chemicals, Uppsala, Sweden). Desalted fractions were flash frozen and stored in liquid nitrogen. Protein concentration was determined using the Pierce BCA protein concentration assay kit (Thermo Fisher Scientific, Rockford, Ill.)

### Example 4,

### Initial Activity Assays

Initial activity assays were conducted using thin layer chromatography (TLC) and a gas chromatography (GC) assay similar to that described previously (29). To test activity, 0.3 mg of protein was added to a reaction vessel along with 200 μM palmitoleyl-CoA and 800 μM NADPH, NADH, NADP⁺, NAD⁺ in reaction buffer containing 20 mM Tris-HCl pH 7.0 and 50 mM NaCl. Reactions were allowed to proceed for 1 hr before quenching by the addition of 2 mL of hexane. The hexane water mixture was vortexed vigorously for 30 seconds before centrifuging to separate phases. The hexane phase was removed to a clean container and the solvent was removed under a stream of argon. The resulting residue was resuspended in 100 μL of hexane and spotted on a TLC silica plate along with 5 μL each of palmitoleyl alcohol (10 mg/mL) and cis-11-hexadecenal (10 mg/mL) standards. The TLC plate was developed in a 2:15:90 volumetric ratio of glacial acetic acid:ethyl ether:hexane. After development, visualization was performed in a sealed jar with iodine crystals for 10 min. The TLC results were verified by GC analysis of the sample prepared in the same way and compared to retention times of known standards.

### Example 5

### pH Studies

Optimal pH was determined by assaying over a range of pH values from 5.5 to 9.0. A buffer composed of 50 mM MES, 50 mM MOPS, 50 mM TAPS, 150 mM NaCl, 0.5 mg/mL BSA was made and the pH adjusted by adding either NaOH or HCl. Assays were conducted using the NADPH continuous spectrophotometric assay described below.

### Example 6

### Continuous Spectrophotometric NADPH Assay

All assays were conducted in a total volume of 1 mL. A buffer containing 50 mM NaCl, 20 mM Tris-HCl pH 7.0, and 0.5 mg/mL BSA was prepared along with a 1 mM stock of aldehyde dissolved in dimethyl sulfoxide (DMSO) or a 0.1 mM stock of the acyl-CoA, and 2.0 trig/mL NADPH. All components including protein were degassed in sealed vials and placed under an argon atmosphere. NADPH was degassed as the solid prior to the addition of degassed buffer. Each assay was conducted by adding 75 μL of the NADPH stock, 58 μg protein for acyl-CoA assays or 15 μg protein for aldehyde assays, varying concentrations of aldehyde or acyl-CoA and buffer to bring the final volume to 1 mL. Each sample was continuously monitored for the decrease of NADPH at 340 nm on a Varian 50 Bio UV-visible spectrophotometer (Walnut Creek, Calif.). Initial rates were calculated in Excel using the linear initial rates of reaction obtained from the spectrophotometric assays by obtaining the slope from the best. In line and calculating nmol of NADPH oxidized per second. (Microsoft, Redmond, Wash.). These initial rates were used to calculate Kₘ and Vₘₐₓ values using the Igor Pro software package (Wavemetrics, Lake Oswego, Oreg.) fitting the initial rates to the Hill equation (31). NADPH specific activity assays were conducted identically as described above using a fixed 60 μM concentration of the various aldehyde substrates or 6 μM of the various acyl-CoA substrates.

### Example 7

### Continuous Spectrophotometric DTNB Assay

Buffers and solutions were prepared as described above for NADPH assays. Each assay was conducted by adding 75 μL of the 2 mg/mL NADPH stock solution in buffer, 58 μg of protein, and 10 μL of a 10 mg/mL solution of DTNB in DMSO, varying concentrations of acyl-CoA and buffer to bring the volume to 1 mL. Reduction of acyl-CoA substrate was monitored by following the increase of the 2-nitro-5-thiobenzoate (NTB²⁻) dianion concentration at 412 nm. Initial rates were calculated in Excel (Microsoft, Redmond, Wash.) using the linear initial rates of reaction obtained from the spectrophotometric assays by obtaining the slope from the best fit line and calculating nmol of NTB²⁻ dianion formed per second using the extinction coefficient of the NTB²⁻ dianion of 14150 M⁻¹ cm⁻¹. These initial rates were used to calculate the apparent K, and Vₘₐₓ values using the Igor Pro (Wavemetrics, Lake Oswego, Oreg.) software package fitting the initial rates to the Hill equation (Equation 1) where v is the initial velocity, Vₘₐₓ is the maximum calculated velocity, [S]ⁿ is the concentration of substrate and n is the Hill coefficient, K_{0.5}ⁿ is the approximation of Kₘ or the approximate substrate concentration at which half of Vₘₐₓ is obtained at a specific value of the Hill coefficient n. In the case of enzyme inhibition, a modified version of the Hill equation allowing for cooperative inhibition (Equation 2) where all of the coefficients are defined as for the Hill equation and the additional [i]ⁿ is the concentration of inhibitor and n is the Hill coefficient. The Kᵢⁿ is the approximate concentration of inhibitor it takes to double the K_{0.5} at a specific value of the Hill coefficient n.

$$\begin{matrix}v = \frac{V {max [ S ]}^{n}}{{K}_{0.5}^{n} + {[ S ]}^{n}} & Equation 1 \\ v = \frac{V {max [ S ]}^{n}}{{K}_{0.5}^{n} ( 1 + \frac{{[ i ]}^{n}}{{K}_{i} n} ) + {[ S ]}^{n}} & Equation 2\end{matrix}$$

### Example 8

### Verification of Activity without MBP Tag

To determine activity of the enzyme with the maltose binding protein (MBP) removed, 500 μg of protein was digested with 10 units of Factor Xa. (New England Biolabs, Ipswich, Mass.), according to the manufacturers prescribed protocol. The resulting protein was used in a set of assays conducted according to the DTNB protocol previously described. The kinetic curve produced was compared to the established curves.

### Example 9

### Determination of Quaternary Structure

Three mg of purified protein was desalted into a buffer containing 150 mM NaCl and 20 mM Tris-HCl pH 7.0. This protein was loaded onto a size exclusion column (High Load 2660 Superdex 200 GE Healthcare) equilibrated with a buffer containing 20 mM Tris-HCl pH 7.0 and 150 mM NaCl along with standards of known native molecular weight using the GE high molecular weight standard kit (GE Healthcare, Uppsala, Sweden) to determine the size of the resulting protein and run at 0.7 mL/min flow rate.

### Example 10

### Methods of Use

The present invention provides for the production of fatty alcohols from their corresponding fatty acyl-CoA, in a single biosynthetic step. Accordingly, the present invention also provides for new methods of making and using fatty alcohols to make products in cosmetics, pharmaceuticals, and industrial applications. Methods of using fatty alcohols may include (i) producing fatty alcohols by in single biosynthetic step by methods described herein, and (ii) using the produced fatty alcohols in ways standard in the arts of cosmetics, pharmaceuticals, or other arts known to use fatty alcohols. Uses of fatty alcohols may depend on the length of the carbon chain and the mixture of carbon chain lengths. Optionally, smaller carbon chains (C8-C18) may be preferable for use as surfactants in a large number of cosmetic and industrial processes (Knout, J., and Richtler, H. J. (1985) Trends in industrial uses of palm and lauric oils, J Am Oil Chem Soc 62, 317-327.). Alternatively, it may be preferable to use very long chain fatty alcohols (VLFA) (C20-C34) as pharmaceutical agents. For example, VLFAs may be useful in reducing by the symptoms of angiogenic diseases (Duliens, S. P. J., Mensink, R. P., Bragt, M. C. E., Kies, A. K., and Plat, J. (2008) Effects of emulsified policosanols with different chain lengths on cholesterol metabolism in heterozygous LDL receptor-deficient mice, J. Lipid Res 49, 790-796.). Wax esters may be formed from the condensation of a any alcohol with a an acyl CoA (Wälternann, M., Stöveken, T., and Steinbüchel, A. (2007) Key enzymes for biosynthesis of neutral lipid storage compounds in prokaryotes: Properties, function and occurrence of wax ester synthaseslacyl-CoA:diacylglycerol acyltransferases, Biochimie 89, 230-242.).

Alternatively, fatty alcohols can be used as directly in compounds. For example, fatty alcohols may be used directly without further modification. For further example, fatty alcohols produced by a single biosynthetic step, as described herein, may be used in cosmetics and pharmaceuticals. The fatty alcohols may be purified or substantially purified, prior to addition to other compounds, such as, cosmetics and pharmaceuticals.

The present invention also provides for methods of producing wax esters from fatty acid alcohols that were produced in a single biosynthetic step, as described herein. Methods of making wax esters from fatty alcohols may include (i) producing fatty alcohols by in single biosynthetic step by methods described herein, and (ii) using the produced fatty alcohols in ways standard in the art to produce was esters. Wax esters are essential compounds in many cosmetics and pharmaceuticals where they mimic natural human sebaceous gland secretions (Cheng, J. B., and Russell, D. W. (2004) Mammalian Wax Biosynthesis, J Biol Chem 279, 37789-37797). Wax esters also make excellent lubricants for use in high temperature applications (Bell, E. W., Gast, L. E., Thomas, F. L., and Koos, R. E. (1977) Sperm oil replacements: Synthetic wax esters from selectively hydrogenated soybean and linseed oils, J Am Oil Chem Soc 54, 259-263). The original source of wax ester compounds was the sperm whale; however, following the worldwide ban on whaling, plant wax ester sources have become critical. Currently, the major source of these compounds is the jojoba plant, which produces large amounts of wax esters in its seedpods. Wax esters may be synthesized from fatty acids and fatty alcohols using lipase enzymes, but the use of this method is currently not cost effective due to the low availability and high cost of fatty alcohols (Trani, M., Ergan, F., and André, G. (1991) Lipase-catalyzed production of wax esters, J Am Oil Chem Soc 68, 20-22).

### Example 11

Duel Reduction in a Sing Biosynthetic Step

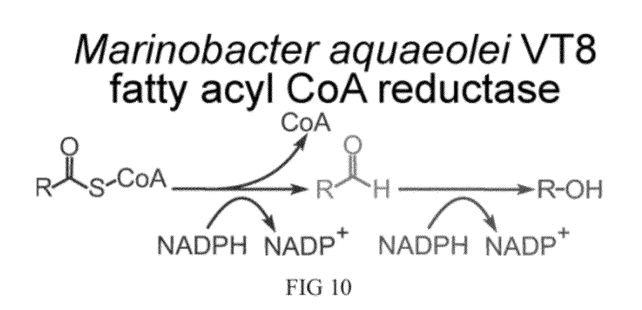

Without limiting the invention, FIG. 10 shows a two-step dual reduction of a fatty acyl-CoA to a corresponding alcohol, carried out in a single biosynthetic step. Accordingly, the present invention provides for a method of making a product by first producing a fatty alcohol from a corresponding fatty acyl-CoA using an enzyme of the present invention to carry out a dual reduction of the fatty acyl-CoA to the fatty alcohol. The fatty alcohol may be the final product. Optionally, the fatty alcohol may then be used to make products including pharmaceuticals, cosmetics, lubricants, and wax esters.

We claim:

1. A composition of matter, comprising an isolated bacterial enzyme capable of reducing a fatty acyl-CoA to a corresponding fatty alcohol in a single biosynthetic step.

2. An enzyme of claim 1, further comprising an amino acid sequence at least 50%, 60%, 70%, 80%, 90%, 95%, 98%, 99%, or 100% similar to SEQ ID NO: 1.

3. An enzyme of claim 1 having dual reductase activity.

4. An enzyme of claim 1, further comprising an oligomeric state.

5. An enzyme of claim 1, further comprising a tetrameric form.

6. An enzyme of claim 1, further comprising a bacterial organism.

7. An enzyme of claim 6, wherein the bacterial organism is capable of producing a fatty alcohol.

8. An enzyme of claim 1, further comprising a plasmid.

9. An isolated enzyme of claim 1, further comprising a monomeric, dimeric, trimeric, tetrameric, or other oligomeric form of a protein.

10. An enzyme of claim 1, further comprising an amino acid sequence at least 50%, 60%, 70%, 80%, 90%, 95%, 98%, 99%, or 100% similar to amino acids 1 to 364 of SEQ ID NO: 1.

11. An enzyme of claim 1, further comprising an amino acid sequence at least 50%, 60%, 70%, 80%, 90%, 95%, 98%, 99%, or 100% similar to amino acids 364 to 601 of SEQ ID NO: 1.

12. A composition of matter, comprising an isolated amino acid sequence at least 50%, 60%, 70%, 80%, 90%, 95%, 98%, 99%, or 100% identical to SEQ ID NO: 1.

13. An amino acid sequence of claim 12, further comprising an enzyme capable of reducing a fatty acyl-CoA to a corresponding fatty alcohol in a single biosynthetic step.

14. An amino acid sequence of claim 12, further comprising an enzyme having dual reductase activity.

15. A method of isolating an enzyme of claim 1, comprising at least one of the following steps: (i) cloning a gene encoding an enzyme capable of reducing a fatty acyl-CoA to a corresponding fatty alcohol in a single biosynthetic step, (ii) inserting the cloned gene into a plasmid, (iii) transforming, the plasmid into a suitable host for protein expression, (iv) expressing the protein encoded by the gene, and (v) substantially purifying the protein.

16. A method of making a product, comprising (i) producing a fatty alcohol from a corresponding fatty acyl-CoA using an isolated bacterial enzyme having dual reductase activity to carry out a dual reduction of the fatty acyl-CoA to the corresponding fatty alcohol.

17. The method of claim 16, wherein the fatty alcohol is the product.

18. The method of claim 16, wherein the product is from a group of products consisting of pharmaceuticals, cosmetics, lubricants, and wax esters.


SEQUENCE LISTING (2 sequences)

SEQ ID NO: 1 - protein, 591 aa, Marinobacter aquaeolei VT8
TLKSLKGNIDHVFHLAAVYDMGADEEAQAATNIEGTRAAVQAAEAMGAKHFHHVSSIAAAGLFKGIFREDMFEEAEKLDHPYLRTKHESEKVVREECKVPFRIYRPGMVIGHSETGEMDKVDGPYYFFKMIQKIRHALPQWVPTIGIEGGRLNIVPVDFVVDALDHIAHLEGEDGNCFHLVDSDPYKVGEILNIFCEAGHAPRMGMRIDSRMFGFIPPFIRQSIKNLPPVKRITGALLDDMGIPPSVMSFINYPTRFDTRELERVLKGTDIEVPRLPSYAPVIWDYWERNLDPDLFKDRTLKGTVEGKVCVVTGATSGIGLATAEKLAEAGAILVIGARTKETLDEVAASLEAKGGNVHAYQCDFSDMDDCDRFVKTVLDNHGHVDVLVNNAGRSIRRSLALSFDRFHDFERTMQLNYFGSVRLIMGFAPAMLERRRGHVVNISSIGVLTNAPRFSAYVSSKSALDAFSRCAAAEWSDRNVTFTTINMPLVKTPMIAPTKIYDSVPTLTPDEAAQMVADAIVYRPKRIATRLGVFAQVLHALAPKMGEIIMNTGYRMFPDSPAAAGSKSGEKPKVSTEQVAFAAIMRGIYW

SEQ ID NO: 2 - DNA, 1846 bp, Marinobacter aquaeolie VT8
atgaattatttcctgacaggcggcaccggttttatcggtcgttttctggttgagaaactcttggcgcgcggcggcaccgtgtatgttctggttcgcgagcagtcccaggacaagctggagcggctccgggagcgctggggtgcagacgacaagcaagtgaaggctgtgatcggcgacctcaccagcaaaaaccttggtattgacgcgaaaacgctgaaatcactgaaaggaaatatcgaccacgtattccatcttgccgcggtctacgacatgggcgcagacgaagaagcccaggccgccaccaatatcgaaggcaccagggcggctgttcaggccgccgaagccatgggcgccaagcatttccatcatgtgtcatccatcgcggcagcgggtctgttcaagggtatcttccgggaggatatgttcgaagaagccgagaagcttgatcatccttacctgcgcaccaagcacgaatccgaaaaagttgtgcgtgaagaatgcaaggttccgttccgcatctaccgccctggtatggtcattggccattcggaaaccggcgaaatggacaaggttgacgggccctattacttcttcaagatgattcagaagatccgtcatgcgttgccccagggtgggtgagatcctcaatattttctgcgaggccggccatgccccccgcatgggtatgcgcatcgattcccggatgttcggttttattccgccgtttattcgccagagcatcaagaatctgcctccggtcaagcgcattactggtgcgcttctggatgacatgggcattccgccctcggtgatgtccttcattaattacccgacccgttttgatacccgggagctggagcgggttctgaagggcacagacattgaggtgccgcgtctgccgtcctatgccccggttatctgggactactgggagcgcaatctggacccggacctgttcaaggaccgcaccctcaagggcacggttgaaggtaaggtttgcgtggtcaccggcgcgacctcgggtattggcctggcaacggcagagaagctggcagaggccggtgccattctggtcattggtgcgcgcaccaaggaaactctggatgaagtggcggccagtctggaggccaagggtggcaacgtgcatgcgtaccagtgcgacttttcggacatggacgactgcgaccgctttgtgaagacggtgctggataatcacggccacgtggatgtactggtgaataacgcgggtcgctccatccgccgctcgctggcgttgtcttttgaccggttccacgattttgagcggaccatgcagctgaactactttggctccgttcggctgatcatgggctttgcgccagccatgctggagcgtcgccgcgggcacgtggtgaatatttcttccatcggggtacttaccaacgctccgcgtttctcggcctatgtctcctcgaaatccgcactggacgcgttcagccgctgtgccgctgcagaatggtcggatcgcaacgtgaccttcaccaccatcaacatgccgttggtgaaaacgccgatgatcgcgcccaccaagatctacgattccgtgccgacgctgacgccggatgaagccgcccagatggtggcggatgcgattgtgtaccggcccaagcgcattgccacccgtcttggcgtgttcgcgcaggttctgcatgcgctggcaccgaagatgggtgagatcattatgaacactggctaccggatgttcccggattctccagcagccgctggcagcaagtccggcgaaaagccgaaagtctctaccgagcaggtggcctttgcggcgattatgcgggggatatactggtaa